(19) United States
(12) Patent Application Publication
(10) Pub. No.: US 2004/0237130 A1
(43) Pub. Date: Nov. 25, 2004

(54) Embryogenic culture initiation of douglas-fir by maltose

(75) Inventors: Pramod K. Gupta, Federal Way, WA (US); Diane G. Holmstrom, Sumner, WA (US); Doris Budworth, Puyallup, WA (US)

Correspondence Address: WEYERHAEUSER COMPANY, INTELLECTUAL PROPERTY DEPT., CH 1J27, P.O. BOX 9777, FEDERAL WAY, WA, 98063, US

(73) Assignee: Weyerhaeuser Company

(21) Appl. No.: 10/819,466

(22) Filed: Apr. 7, 2004

Related U.S. Application Data
(60) Provisional application No. 60/468,304, filed on May 5, 2003.

(51) Int. Cl. A01H 1/00; A01H 1/02; C12N 5/00; C12N 5/02

(52) U.S. Cl. 800/260

(57) ABSTRACT

In one aspect, the present invention provides methods for producing Douglas-fir somatic embryos. In one aspect, the methods of the invention comprise the step of culturing Douglas-fir explants in, or on, an initiation medium comprising a principal carbohydrate source selected from the group consisting of maltose, glucose, and a combination thereof, to form embryonal suspensor masses comprising early-stage somatic embryos. In another aspect, the methods of the invention additionally comprise the step of culturing Douglas-fir embryonal suspensor masses in maintenance medium comprising a principal carbohydrate source selected from the group consisting of maltose, glucose, and a combination thereof, to form an established embryonal suspensor mass comprising early-stage somatic embryos.

### CROSS REFERENCE TO RELATED APPLICATION

The present application claims the benefit of U.S. Provisional Application No. 60/468,304, filed May 5, 2003.

### FIELD OF THE INVENTION

The present invention relates to methods for producing Douglas fir somatic embryos.

### BACKGROUND OF THE INVENTION

The demand for coniferous trees, such as pines and firs, to make wood products continues to increase. One proposed solution to this problem is to identify individual trees that possess desirable characteristics, such as a rapid rate of growth, and produce numerous, genetically identical, clones of the superior trees by somatic cloning.

Somatic cloning is the process of creating genetically identical trees from tree tissue other than the male and female gametes. In one approach to somatic cloning, plant tissue is cultured in an initiation medium that includes hormones, such as auxins and/or cytokinins, to initiate formation of embryogenic cells, such as embryonic suspensor masses, that are capable of developing into somatic embryos. The embryogenic cells are then further cultured in a maintenance medium that promotes establishment and multiplication of the embryogenic cells. The multiplied embryogenic cells are then cultured in a development medium that promotes development of conifer somatic embryos which can, for example, be placed within artificial seeds and sown in the soil where they germinate to yield conifer seedlings. The seedlings can be transplanted to a growth site for subsequent growth and eventual harvesting to yield lumber, or wood-derived products.

A continuing problem with somatic cloning of Douglas-fir embryos is stimulating efficient initiation and establishment of Douglas-fir embryogenic cells that are capable of producing somatic embryos. The present invention provides methods that satisfy this need.

### SUMMARY OF THE INVENTION

In one aspect, the present invention provides methods for producing Douglas-fir somatic embryos. In one aspect, the methods of the invention comprise the step of culturing Douglas-fir explants in, or on, an initiation medium comprising a principal carbohydrate source selected from the group consisting of maltose, glucose, and a combination thereof, to form embryonal suspensor masses comprising early-stage somatic embryos. The concentration of maltose or glucose, or combination thereof, in the initiation medium may be from about 5 g/L to about 60 g/L, such as from about 20 g/L to about 50 g/L or from about 30 g/L to about 40 g/L. In some embodiments, the Douglas-fir explants are cultured in, or on, the initiation medium for a period of from about 4 weeks to about 12 weeks, such as from about 5 weeks to about 10 weeks, such as about 6 weeks.

In another aspect, the methods of the invention additionally comprise the step of culturing Douglas-fir embryonal suspensor masses in maintenance medium comprising a principal carbohydrate source selected from the group consisting of maltose, glucose, and a combination thereof, to form an established embryonal suspensor mass comprising early-stage somatic embryos. An established embryonal suspensor mass is growing and multiplying. Embryonal suspensor masses that do not become established turn brown and die. The concentration of maltose or glucose, or combination thereof, in the maintenance medium may be from about 5 g/L to about 60 g/L, such as from about 20 g/L to about 50 g/L or from about 30 g/L to about 40 g/L. In some embodiments, the Douglas-fir explants are cultured in, or on, the maintenance medium for a period up to about 6 months.

In some embodiments of the methods, at least 27% of Douglas-fir explants form an embryonal suspensor mass. The methods of the invention produce an increased number of initiated Douglas-fir explants compared to a method in which the initiation medium does not contain maltose or glucose, or a combination thereof, as the principal carbohydrate source. For example, the number of embryonal suspensor masses produced using an initiation medium of the invention is at least about 51% higher than the number of embryonal suspensor masses produced using an identical initiation medium but comprising sucrose as the principal carbohydrate source.

In some embodiments of the methods, at least 18% of the Douglas-fir embryonal suspensor masses form an established embryonal suspensor mass. The methods of the invention produce an increased number of established Douglas-fir embryonal suspensor masses compared to a method in which the maintenance medium does not contain maltose or glucose, or a combination thereof, as the principal carbohydrate source. In some embodiments, the number of established Douglas-fir embryonal suspensor masses produced according to the methods of the invention is at least about 65% higher than the number of established Douglas-fir embryonal suspensor masses produced using an identical maintenance medium but comprising sucrose as the principal carbohydrate source.

The methods of the present invention are useful, for example, for preparing Douglas fir somatic embryos that can be further characterized, such as by genetic or biochemical means, and/or can be germinated to yield Douglas-fir plants that can be grown into mature Douglas-fir trees, if so desired. Thus, for example, the methods of the invention can be used to produce clones of individual Douglas-fir trees that possess one or more desirable characteristics, such as a rapid growth rate or improved wood quality. For example, a population of Douglas-fir somatic embryos of the invention can be used to produce a stand, or forest, of Douglas-fir trees possessing one or more desirable characteristics, such as a rapid growth rate or improved wood quality. The trees can be utilized to produce wood products.

### DETAILED DESCRIPTION OF THE PREFERRED EMBODIMENT

Unless specifically defined herein, all terms used herein have the same meaning as they would to one skilled in the art of the present invention.

As used herein, the term “explant” refers to a piece of tissue from a donor plant. Typical explants used in accordance with the methods of the invention are fertilized embryos from immature seeds.

As used herein, the term “embryonal suspensor mass” or “ESM” refers to a white, translucent, and mucilaginous mass consisting of embryos at varying early stages of development, each containing an embryonal head and suspensor system.

As used herein, the term “established embryonal suspensor mass” refers to an embryonal cell mass that is growing and multiplying. Embryonal suspensor masses that do not become established turn brown and die. An established embryonal suspensor mass typically weighs between 250 mg and 500 mg or that measures 1-2 centimeter in diameter after several subcultures.

As used herein, the term “early-stage somatic embryo” refers to an embryo at the pre-dome stage of development. The term “cotyledonary somatic embryo” refers to an embryo that possesses at least one cotyledon.

As used herein, the term “principal carbohydrate source” refers to the carbohydrate source that is present in the highest concentration.

Unless stated otherwise, all concentration values that are expressed as percentages are weight per volume percentages.

In one aspect, the present invention provides methods for producing Douglas-fir somatic embryos, comprising the step of culturing Douglas-fir explants in, or on, an initiation medium comprising a principal carbohydrate source selected from the group consisting of maltose, glucose, and a combination thereof, to form embryonal suspensor masses comprising early-stage somatic embryos.

An example of explants useful in the practice of the present invention are pre-dome and dome stage embryos from Douglas-fir cone seeds, as described in EXAMPLE 1. Typically, the seeds are surface sterilized before removing the embryos, which are then cultured on, or in, an initiation medium. According to the invention, the initiation medium contains maltose or glucose, or a combination thereof, as the sole, or principal, metabolizable sugar source. Prior art initiation media for Douglas-fir explants use sucrose as a carbohydrate source. Sucrose is a disaccharide composed of covalently linked glucose and fructose molecules. Glucose is a good carbohydrate source for biosynthesis, in contrast to fructose. Maltose is a disaccharide composed of two glucose units. Thus, the use of maltose as the principal carbohydrate source provides twice the amount of glucose per carbohydrate unit than that provided by sucrose. The concentration of the principal carbohydrate source in the initiation medium may be from about 5 g/L to about 60 g/L, such as from about 20 g/L to about 50 g/L or from about 30 g/L to about 40 g/L.

The initiation medium may be a semi-solid medium or a solid medium. The initiation medium typically includes inorganic salts and organic nutrient materials. The osmolality of the initiation medium is typically about 160 mM/kg or even lower, but it may be as high as 170 mM/kg. The initiation medium typically includes growth hormones. Examples of hormones that can be included in the initiation medium are auxins (e.g., 2,4-dichlorophenoxyacetic acid (2,4-D)) and cytokinins (e.g., 6-benzylaminopurine (BAP)). Auxins can be utilized, for example, at a concentration of from about 1 mg/L to about 200 mg/L. Cytokinins can be utilized, for example, at a concentration of from about 0.1 mg/L to about 50 mg/L.

The initiation medium may contain gellan gum, typically present at a concentration of about 1 g/L to about 2 g/L. The initiation medium may contain an absorbent composition, especially when very high levels of growth hormones are used. The absorbent composition can be any composition that is not toxic to the cells at the concentrations utilized in the practice of the present methods, and that is capable of absorbing growth-promoting hormones, and toxic compounds produced by the plant cells during embryo development, that are present in the medium. Non-limiting examples of useful absorbent compositions include activated charcoal, soluble poly(vinyl pyrrolidone), insoluble poly(vinyl pyrrolidone), activated alumina, and silica gel. The absorbent composition may be present in an amount, for example, of from about 0.1 g/L to about 5 g/L.

An example of an initiation medium useful in the practice of the present invention is medium BM₁, set forth in EXAMPLE 1 herein.

Douglas-fir explants are typically cultured in, or on, an initiation medium for a period of from 4 weeks to 12 weeks, such as from 5 weeks to 10 weeks, or such as about 6 weeks, at a temperature of from 10° C. to 30° C., such as from 15° C. to 25° C., or such as from 20° C. to 23° C.

In another aspect, the present invention provides methods for producing Douglas-fir somatic embryos, comprising the steps of: (a) culturing Douglas-fir explants in, or on, an initiation medium comprising a principal carbohydrate source selected from the group consisting of maltose, glucose, and a combination thereof, to form embryonal suspensor masses comprising early-stage somatic embryos, and (b) culturing the embryonal suspensor masses in, or on, a maintenance medium comprising a principal carbohydrate source selected from the group consisting of maltose, glucose, and a combination thereof, to form established embryonal suspensor masses. An established embryonal suspensor mass is one that is growing and multiplying. Embryonal suspensor masses that do not become established turn brown and die.

The composition of the initiation medium is as described above. Typically, maltose or glucose, or a combination thereof, is the sole, or principal, metabolizable sugar source in the maintenance medium. The concentration of maltose or glucose, or combination thereof, in the maintenance medium may be from about 5 g/L to about 60 g/L, such as from about 20 g/L to about 50 g/L or from about 30 g/L to about 40 g/L.

The maintenance medium may be a solid medium, or it may be a liquid medium, which can be agitated to promote growth and multiplication of the ESMs. The osmolality of the maintenance medium is typically higher than the osmolality of the initiation medium, typically in the range of 180-400 mM/kg. The maintenance medium may contain nutrients that sustain the embryogenic tissue, and may include hormones, such as one or more auxins and/or cytokinins, that promote cell division and growth of the embryogenic tissue. Typically, the concentrations of hormones in the maintenance medium are lower than their concentration in the initiation medium. An example of a suitable maintenance medium is medium BM₂ set forth in EXAMPLE 1 herein.

Douglas-fir ESM are typically cultured in, or on, a maintenance medium for a period of up to 6 months, at a temperature of from 10° C. to 30° C., such as from 15° C. to 25° C., or such as from 20° C. to 23° C. When using solid maintenance medium, ESMs are typically subcultured about every 2 weeks, when using liquid maintenance medium, ESMs are typically subcultured weekly.

In some embodiments, the invention provides methods of producing Douglas-fir somatic embryos, comprising the steps of: (a) culturing Douglas-fir explants in, or on, an initiation medium comprising a principal carbohydrate source selected from the group consisting of maltose, glucose, and a combination thereof, to form embryonal suspensor masses comprising early-stage somatic embryos; (b) culturing the embryonal suspensor masses in, or on, a maintenance medium comprising a principal carbohydrate source selected from the group consisting of maltose, glucose, and a combination thereof, to form established embryonal suspensor masses comprising early-stage somatic embryos; (c) culturing the early-stage somatic embryos from established embryonal suspensor masses in, or on, a singulation medium to form singulated early-stage somatic embryos; and (d) culturing the singulated early-stage somatic embryos in, or on, a development medium to form cotyledonary somatic embryos.

According to this embodiment, established ESM containing early-stage somatic embryos may further be cultured in, or on, a singulation medium to form singulated embryos. Suitable singulation media typically do not include growth-promoting hormones, such as auxins and cytokinins, but generally include the hormone abscisic acid. Abscisic acid is a sesquiterpenoid plant hormone that is implicated in a variety of plant physiological processes (see, e.g., Milborrow (2001) J. Exp. Botany 52: 1145-1164; Leung & Giraudat (1998) Ann. Rev. Plant Physiol. Plant Mol. Biol. 49: 199-123). When abscisic acid is utilized in the development medium, it is typically utilized at a concentration in the range of from about 1 mg/L to about 200 mg/L. Typically, the singulation medium is a liquid medium and contains nutrients that sustain the embryogenic tissue. The singulation medium may contain maltose, glucose, or a combination thereof, as the principal metabolizable sugar source. The osmolality of the singulation medium can be adjusted to a value that falls within a desired range, such as from about 150 mM/kg to about 250 mM/kg. Typically, an osmolality of 200 mM/kg or higher is advantageous. An example of a suitable development medium is medium BM₃ set forth in EXAMPLE 1 herein.

Douglas-fir early-stage somatic embryos may be cultured in, or on, a singulation medium with weekly subcultures for a period of from 1 week to 3 weeks, at a temperature of from 10° C. to 30° C., such as from 15° C. to 25° C., or such as from 20° C. to 23° C.

After singulation, early-stage somatic embryos may be cultured on a development medium to form cotyledonary somatic embryos. The development medium is typically a solid medium, although the development medium can be a liquid medium. The development medium typically contains nutrients that sustain the embryogenic tissue. Maltose may be included in the development medium as the principal or sole source of sugar for the embryogenic tissue. Useful maltose concentrations are within the range of from about 1% to about 2.5%.

Suitable development media typically do not include growth-promoting hormones, such as auxins and cytokinins, but may include the hormone abscisic acid. When abscisic acid is utilized in the development medium, it is typically utilized at a concentration in the range of from about 1 mg/L to about 200 mg/L.

The development medium may contain gellan gum, typically present at a concentration of up to about 0.35%. The osmolality of the development medium can be adjusted to a value that falls within a desired range, such as from about 250 mM/kg to about 450 mM/kg. Typically, an osmolality of 350 mM/kg or higher is advantageous. An example of a suitable development medium is medium BM₃ set forth in EXAMPLE 1 herein.

Douglas-fir singulated early-stage embryos may be cultured in, or on, a development medium to form cotyledonary somatic embryos for a period of from 4 weeks to 14 weeks, such as from 8 week to 12 weeks, or such as about 12 weeks, at a temperature of from 10° C. to 30° C., such as from 15° C. to 25° C., or such as from 20° C. to 23° C.

The cotyledonary somatic embryos produced using the methods of the invention can optionally be germinated to form Douglas-fir plants which can be grown into Douglas-fir trees, if desired. Alternatively, the mature embryos may be disposed within artificial seeds for subsequent germination. The cotyledonary Douglas-fir somatic embryos can be germinated, for example, on a solid germination medium, such as medium BM₅ medium set forth in EXAMPLE 1 herein.

In some embodiments, Douglas-fir cotyledonary somatic embryos are transferred to a stratification medium for a cold treatment prior to germination. Typically, the stratification medium is similar to germination medium, but lacks agar. Cotyledonary somatic embryos are typically cultured in, or on, a stratification medium in the dark for a period of from 3 weeks to 6 weeks, such as about 4 weeks, at a temperature of from 1° C. to 10° C., such as from 1° C. to 8° C.

The germinated plants can be transferred to soil for further growth. For example, the germinated plants can be planted in soil in a greenhouse and allowed to grow before being transplanted to an outdoor site. Typically, the mature Douglas-fir somatic embryos are illuminated to stimulate germination. Typically, all the steps of the methods of the invention, except germination, are conducted in the dark.

In some embodiments of the methods, at least 27% (such as at least 40% or at least 60%) of Douglas-fir explants form an embryonal suspensor mass. The methods of the invention produce an increased number of embryonal suspensor masses initiated from Douglas-fir explants compared to a method in which the initiation medium does not contain maltose or glucose, or a combination thereof, as the principal source of carbohydrates. For example, the number of embryonal suspensor masses produced according to the methods of the invention is at least about 51% higher (such as about 60% higher or about 80% higher) than the number of embryonal suspensor masses produced using an identical initiation medium but comprising sucrose as the principal carbohydrate source.

In some embodiments of the methods, at least 18% (such as at least 25% or at least 40%) of the Douglas-fir embryonal suspensor masses form an established embryonal suspensor mass. The methods of the invention produce an increased number of established Douglas-fir embryonal suspensor masses compared to a method in which the maintenance medium does not contain maltose or glucose, or a combination thereof, as the principal carbohydrate source. In some embodiments, the number of established Douglas-fir embryonal suspensor masses produced according to the methods of the invention is at least about 65% higher (such as about 75% higher or about 90% higher) than the number of established Douglas-fir embryonal suspensor masses produced using an identical maintenance medium but comprising sucrose as the principal carbohydrate source.

The methods of the invention can be used, for example, to produce clones of individual Douglas-fir trees that possess one or more desirable characteristics, such as a rapid growth rate. Thus, in one aspect, the present invention provides methods for producing a population of genetically-identical, mature Douglas-fir somatic embryos. Any of the methods described herein can be used to produce populations of genetically-identical, mature somatic Douglas-fir embryos.

The following examples merely illustrate the best mode now contemplated for practicing the invention, but should not be construed to limit the invention.

### EXAMPLE 1

This Example shows a comparison of Douglas-fir embryonal suspensor mass initiation and establishment frequencies obtained using initiation and maintenance media containing either maltose or sucrose as the principal carbohydrate source.

Cones were collected from four mother tree crosses (full-sib families) at a time when the embryos were in the pre-dome and dome stage of development. Cones were transferred on blue ice and stored in plastic bags in a cold room at 0-4° C. until needed for dissections. Seeds were removed from cones and dissected on the same day. After the seeds were removed, they were placed into a beaker containing pre-sterilized water.

Seeds were divided into aliquots of 60-80 seeds. Each aliquot was placed into a separate pre-sterilized tea strainer before undergoing the following procedure. First, seeds were agitated in 10% Liqui-nox (+2 droppers full Tween-20 per liter) for 10 minutes. After rinsing the seeds in deionized water (18 megaohms) for 30 minutes, the tea strainers were exposed to 20% (v/v) H₂O₂ for 10 minutes in a fume hood on a rotary shaker (175 rpm). The H₂O₂ was diluted just before use with sterile, deionized water. Following application of the H₂O₂, each strainer was placed into a separate beaker of sterile deionized water, covered with foil, and moved into the laminar flow hood. All subsequent steps were carried out in the laminar flow hood. Each tea strainer was rinsed by agitating 4-8 times in each of 5 separate portions of sterile deionized water. The contents of each tea strainer were then dumped into a large petri dish containing a sterile filter paper and a few millimeters of sterile water (to help keep seeds from drying). Seeds were placed into 60×15 mm sterile petri plates containing 0.2 ml sterile deionized water each. Those seeds not dissected after sterilization were thrown out at the end of the day.

Dissections were done in clean laminar flow hoods. Clean gloves were worn throughout the preparatory steps and dissection. Sterile forceps and scalpels were used to carefully split the seed, remove the female gametophyte and nucellus, and to split the female gametophyte. The embryo suspensor was pulled out, but kept attached to (or touching) the female gametophyte. Embryo and gametophyte were placed unto initiation medium BM₁ so that the embryo was visible through the petri dish lid.

Tables 1 and 2 set forth the compositions of media useful for producing Douglas-fir somatic embryos.

TABLE 1
Composition of Basal Medium (BM) for
Douglas-fir Embryogenic Culture

| Constituent | Concentration (mg/L) |
|---|---|
| KNO₃ | varies |
| MgSO₄.7H₂O | 400 |
| KH₂PO₄ | 340 |
| CaCl₂.4H₂O | 200 |
| KI | 1 |
| H₃BO₃ | 5 |
| MnSO₄.H₂O | 20.8 |
| ZnSO₄.7H₂O | 8 |
| NaMoO₄.2H₂O | 0.2 |
| CuSO₄.5H₂O | 0.024 |
| CoCl₂.6H₂O | 0.025 |
| Ca(NO₃)₂.2H₂O | varies |
| FeSO₄.7H₂O | 27.85 |
| Na₂EDTA | 37.25 |
| Nicotinic acid | 0.5 |
| Pyridoxine.HCl | 0.5 |
| Thiamine.HCl | 1 |
| Glycine | 2 |
| Casamino acids | 500 |
| L-Glutamine | varies |
| Myo-Inositol | varies |
| Sugar | varies |
| pH adjusted to 5.7 | |

TABLE 2
Compositions of Douglas-fir Embryogenic Culture Media

| | BM₁ | BM₂ | BM₃ | BM₄ | BM₅ |
|---|---|---|---|---|---|
| Constituent | Initiation | Maintenance | Singulation | Development | Germination |
| KNO₃ | 1250 | 1250 | 1050 | 2500 | 1170 |
| Ca(NO₃)₂.2H₂O | — | — | 200 | — | 220 |
| Myo-Inositol | 1000 | 5000 | 100 | 100 | 100 |
| L-Glutamine | 450 | 1000 | 1000 | 750 | — |
| L-Proline | — | — | — | 100 | — |
| L-Asparagine | — | — | — | 100 | — |
| L-Arginine | — | — | — | 50 | — |
| L-Alanine | — | — | — | 20 | — |
| L-Serine | — | — | — | 20 | — |
| Maltose | 15,000 | 30,000 | 20,000 | 25,000 | — |
| Sucrose | — | — | — | — | 20,000 |
| PEG 8000 | — | — | — | 190,000 | — |
| 2,4-D | 110 | 1.1 | — | — | — |
| N6- | 45 | 0.22 | — | — | — |
| Benzyladenine | | | | | |
| Kinetin | 43 | 0.22 | — | — | — |
| Abscisic acid | — | — | 10/5/5 | 10 | — |
| Gibberellin 4/7 | — | — | — | 7.5 | — |
| Activated | 2500 | — | — | 1000 | 2500 |
| charcoal | | | | | |
| Agar | — | 5000* | — | — | 8000 |
| Gelrite | 1800 | — | — | — | — |

pH of all media are adjusted to 5.7
*not used for liquid media
**tissue culture agar

Initiation: Half of the embryos and gametophytes (Group 1) from each cross were placed on a semi-solid BM₁ initiation medium (Table 2), the other half (Group 2) were placed on semi-solid BM₁ initiation medium in which the maltose was replaced with sucrose (15000 mg/L). The explants were held in an environment at 22°-25 C with a 24 hour dark photoperiod for a time of 5-6 weeks. The length of time depends on the particular genotype being cultured. Embryonal suspensor masses (ESM) (White, translucent, and mucilaginous) were observed beginning 2-3 weeks after inoculation. Microscopic examination typically reveals numerous early stage embryos associated with the mass.

The number of ESM cultures initiated from explants for each group is shown in Table 3. Initiation is defined as growth of an embryonal suspensor mass from an explant. Overall, the percentage of ESMs initiated from explants was 27% for Group A and 18% for Group B. Initiation improved for each of the 4 crosses tested when explants were cultured on initiation medium containing maltose (Group A). Overall, the number of explants initiated increased by 51% for explants cultured on initiation medium containing maltose, compared to explants cultured on initiation medium containing sucrose.

TABLE 3
Initiation Frequencies for Group A and Group B

| Cross | Group | Explants (#) | Initiation (#) | Initiation (%) |
|---|---|---|---|---|
| 1 | A | 136 | 63 | 46 |
| | B | 138 | 42 | 30 |
| 2 | A | 108 | 30 | 28 |
| | B | 108 | 20 | 19 |
| 3 | A | 110 | 21 | 19 |
| | B | 108 | 14 | 13 |
| 4 | A | 112 | 13 | 12 |
| | B | 111 | 8 | 7 |
| Total | A | 466 | 127 | 27 |
| | B | 465 | 84 | 18 |

Maintenance: After 5-6 weeks, ESMs from Group A were subcultured onto a solid BM₂ maintenance medium (Table 2), and ESMs from Group B were subcultured unto a BM₂ maintenance medium in which the maltose was replaced with sucrose (30000 mg/L). The maintenance medium differs from the initiation medium in that the growth hormones (both auxins and cytokinins) are reduced by at least a full order of magnitude. Osmolality of this medium was typically raised from that of the initiation medium to about 180 mM/kg or higher (typically within the range of about 180-400 mM/kg) by increasing the concentration of myo-inositol to 0.5% w/v. The temperature and photoperiod were again 22°-25 C. with 24 hours in the dark. ESMs were subcultured every 12 days onto fresh maintenance medium.

The number of ESMs established from explants for each group after a total of 12-15 subcultures on maintenance is shown in Table 4. An ESM was considered to be established if it weighed between 250 mg and 500 mg (fresh weight) or if measured 1-2 cms in diameter, after several subcultures. The proportion of established ESMs obtained was 18% for explants in Group A and 11% for explants in Group B. The frequencies of established ESMs improved for each of the 4 crosses tested when explants were cultured on maintenance medium containing maltose (Group A). Overall, the number of established ESMs increased by 51% for explants cultured on maintenance medium containing maltose, compared to explants cultured on maintenance medium containing sucrose.

TABLE 4
Establishment Frequencies for Group A and Group B

| Cross | Group | Explants (#) | Initiation (#) | Initiation (%) |
|---|---|---|---|---|
| 1 | A | 136 | 36 | 26 |
| | B | 138 | 20 | 14 |
| 2 | A | 108 | 11 | 10 |
| | B | 108 | 6 | 6 |
| 3 | A | 110 | 20 | 18 |
| | B | 108 | 12 | 11 |
| 4 | A | 112 | 17 | 15 |
| | B | 111 | 13 | 12 |
| Total | A | 466 | 84 | 18 |
| | B | 465 | 51 | 11 |

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for producing Douglas-fir somatic embryos, comprising the step of culturing Douglas-fir explants in, or on, an initiation medium comprising a principal carbohydrate source selected from the group consisting of maltose, glucose, and a combination thereof, to form an embryonal suspensor mass comprising early-stage somatic embryos.

2. The method of claim 1, wherein the concentration of the principal carbohydrate source in the initiation medium is between about 5 g/L and about 60 g/L.

3. The method of claim 1, wherein the principal carbohydrate source is maltose.

4. The method of claim 1, wherein the explants are cultured in, or on, the initiation medium for a period between about 4 weeks and about 12 weeks.

5. The method of claim 1 further comprising the step of culturing the embryonal suspensor masses in maintenance medium comprising a principal carbohydrate source selected from the group consisting of maltose, glucose, and a combination thereof, to form established embryonal suspensor masses comprising early-stage somatic embryos.

6. The method of claim 5, wherein the concentration of the principal carbohydrate source in the maintenance medium is between about 5 g/L and about 60 g/L.

7. The method of claim 5, wherein the carbohydrate source is maltose.

8. The method of claim 5, wherein the embryonal suspensor masses are cultured in, or on, the maintenance medium for a period between about 6 weeks and about 12 weeks.

9. The method of claim 5 further comprising the steps of (a) culturing the early stage somatic embryos from established embryonal suspensor masses in, or on, a singulation medium to form singulated early-stage somatic embryos; and (b) culturing the singulated early-stage somatic embryos in, or on, a development medium to form cotyledonary somatic embryos.

10. The method of claim 1, wherein at least 27% of the explants produced an embryonal suspensor mass.

11. The method of claim 1, wherein the number of embryonal suspensor masses produced is at least about 50% greater than the number of embryonal suspensor masses produced using an identical initiation medium but comprising sucrose as the principal carbohydrate source.

12. The method of claim 5, wherein at least 18% of the embryonal suspensor masses formed established embryonal suspensor masses.

13. The method of claim 5, wherein the number of established embryonal suspensor masses is at least about 65% greater than the number of established embryonal suspensor masses produced using an identical maintenance medium but comprising sucrose as the principal carbohydrate source.
